(19) United States
(12) Patent Application Publication
(10) Pub. No.: US 2010/0249042 A1
(43) Pub. Date: Sep. 30, 2010

(54) COMPOSITIONS AND METHODS FOR TREATMENT OF EYELASHES AND EYEBROWS

(75) Inventor: Leonard M. Patt, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: ProCyte Corporation, Bothell, WA (US)

(21) Appl. No.: 12/697,048

(22) Filed: Jan. 29, 2010

Related U.S. Application Data
(60) Provisional application No. 61/148,260, filed on Jan. 29, 2009.

(51) Int. Cl. A61K 38/06; A61Q 5/00

(52) U.S. Cl. 514/20.7

(57) ABSTRACT

The fullness and length of eyelashes and eyebrows can be enhanced by topical application of a composition comprising at least one peptide copper complex to an area of skin at the base of the eyelashes and eyebrows. Disclosed are compositions and methods for such treatment.

### CROSS-REFERENCE TO RELATED APPLICATION

This application claims the benefit under 35 U.S.C. §119(e) of U.S. Provisional Patent Application No. 61/148,260, filed Jan. 29, 2009, which application is incorporated herein by reference in its entirety.

### BACKGROUND OF THE INVENTION

1. Field of the Invention

The present invention generally relates to the treatment of eyelashes and eyebrows and, more specifically, to the treatment of eyelashes and eyebrows by topical application of a composition comprising a peptide copper complex.

2. Description of the Related Art

Typically hair on the human scalp grows about 0.3 to 0.4 mm/day or about 6 inches per year. Unlike other mammals, human hair growth and loss is random and not seasonal or cyclic. At any given time, a random number of hairs will be in various stages of growth and shedding. There are three stages of hair growth: anagen, catagen, and telogen.

Anagen is the active phase of the hair. The cells in the root of the hair are dividing rapidly. A new hair is formed and pushes the old hair up the follicle and eventually out. During this phase the hair grows about 1 cm every 28 days. Scalp hair stays in this active phase of growth for 2-6 years. The length and fullness of the hair depends on the length of the anagen growth phase, where fullness is a combination of the number of hair shafts and the thickness of the shafts. The hair on the arms, legs, eyelashes, and eyebrows has a very short active growth phase of about 30-45 days which explains why they are so much shorter than scalp hair.

The catagen phase is a transitional stage and 3% of all hairs are in this phase at any time. This phase lasts for about 2-3 weeks for scalp hair during which time hair growth stops.

Telogen is the resting phase of the hair cycle and accounts for 10-15% of all hairs. This phase lasts for about 100 days for hairs on the scalp and much longer for hairs on the eyebrow, eyelash, arm and leg. During this phase the hair follicle is completely at rest.

The short length of the anagen phase and the long length of the telogen phase are the key differences between eyelash and eyebrow hair and scalp hair in humans.

Copper is essential to vital cellular and enzyme processes required for human health and plays a key role in several of the body's essential enzyme systems needed for tissue repair and other biological responses. These copper-based enzyme systems allow tissue to repair itself, blood vessels to form, wounds to close and inflammation to decrease.

Skin health, dermal wound healing, general soft tissue repair, and hair growth require many of the same biological processes such as reconstitution of an extracellular matrix and increased blood from angiogenesis. Copper is utilized by essentially every cell and organ; resulting in the formation of important copper-dependent enzymes—including cytochrome C oxidase (energy production), superoxide dismutase (antioxidation) and lysyl oxidase (cross-linking of elastin and collagen in skin) (Jackson E M. The Importance of Copper in Tissue Regulation and Repair: A Review. Cosmetic Dermatology 1997; 10(10):35-6; and Milne D B. Copper in clinical practice. Clin Lab News 1993; 19:80-81).

In numerous studies, copper peptides have been shown to promote new blood vessel growth, enhance the expression of growth factors, activate matrix metalloproteases, and stimulate the formation of new collagen, elastin, and glycosaminoglycan components of tissue to accelerate the repair process (Buffoni F, Pino R, Dal Pozzo A. Effect of tripeptide-copper complexes on the process of skin wound healing and on cultured fibroblasts. Archives Internationales de Pharmacodynamie et de Therapie 1995; Vol.:60; Huang P J, Huang Y C, Su M F, Yang T Y, Huang J R, Jiang C P. In Vitro Observations on the Influence of Copper Peptide Aids for the LED Photoirradiation of Fibroblast Collagen Synthesis. Photomed Laser Surg 2007; 25:183-90; Maquart F X, Bellon G, Chaqour B, Wegrowski J, Patt L M, Trachy R E et al. In vivo stimulation of connective tissue accumulation by the tripeptide-copper complex glycyl-L-histidyl-L-lysine-Cu2+ in rat experimental wounds. J Clin Invest 1993; 92:2368-76; Maquart F X, Pickart L, Laurent M, Gillery P, Monboisse J C, Borel J P. Stimulation of collagen synthesis in fibroblast cultures by the tripeptide-copper complex glycyl-L-histidyl-L-lysine-copper(2+). FEBS Lett 1988; 238:343-6; McCormack M C, Nowak K C, Koch R J. The effect of copper tripeptide and tretinoin on growth factor production in a serum-free fibroblast model. Arch Facial Plast Surg 2001; 3:28-32; Oddos T, Jumeau-Lafond A, Ries G. Requirement Of Copper And Tripeptide Glycyl-L-Histidyl-L-Lysine-Cu (GHK) Complex Formation For Collagen Synthesis Activity In Normal Human Dermal Fibroblasts. American Academy of Dermatology 60th Annual Meeting Feb. 22-27, 2002 New Orleans, La. 2002; and Pollard J D, Quan S, Kang T, Koch R J. Effects of copper tripeptide on the growth and expression of growth factors by normal and irradiated fibroblasts. Arch Facial Plast Surg 2005; 7:27-31).

Copper salts alone are usually ineffective or even inhibitory for such applications due to irritation associated with the application of a copper salt to the skin. The copper must be delivered in a biologically acceptable form. As an example, when copper is complexed with a biologically acceptable carrier molecule, such as a peptide, it may then be effectively delivered to the skin and to cells to provide beneficial biological effects such as those mentioned above. As more specific examples, peptide copper complexes that are useful for wound healing and skin health are disclosed in U.S. Pat. Nos. 4,760,051; 4,665,054; 4,877,770; 5,135,913; 5,348,943, 6,927,206 and 6,927,205.

Certain peptide copper complexes have also been found to be effective hair-growth agents. For example, U.S. Pat. Nos. 5,177,061, 5,120,831, 5,214,032, 5,538,945 and 6,017,888 disclose peptide copper complexes which are effective in stimulating the growth of hair in warm-blooded animals. These patents disclose compositions and methods for treating hair loss including hair loss associated with both androgenetic and secondary alopecia due to, for example, chemotherapy. Other patents disclose combinations of copper peptide in liposomes containing ginseng saponins, Glycyrrhiza uralensis, biotin, and cytokines for prevention of hair loss (KR Patent No. 2007017563)

A number of peptide copper complexes have been demonstrated to have stimulatory effects of the growth of hair in mouse models (Trachy R E, Fors T D, Pickart L, Uno H. The hair follicle-stimulating properties of peptide copper complexes. Results in C3H mice. Ann NY Acad Sci 1991; 642:-9; and Trachy R E, Timpe E D, Dunwiddie I. Evaluation of telogen hair follicle stimulation using an in vivo model: results with peptide-copper complexes. In: Maibach H I, editor. Dermatologic research techniques. Boca Raton: CRC Press; 1996. p. 241-54) and in preliminary studies on human (Trachy R E, Patt L M, Duncan G M, Kalis B. Phototrichogram analysis of hair follicle stimulation: a pilot clinical study with a peptide copper complex phototrichogram analysis of hair follicle stimulation: a pilot clinical study with a peptide copper complex. In: Maibach H I, editor. Dermatologic research techniques. Boca Raton: CRC Press; 1996. p. 217-26). These peptide copper complexes were potent stimulators of the transition of telogen follicles to growing anagen follicles when used intradermally or topically. In human volunteers, the peptide copper complex treatment also led to an increase in anagen hair density of the scalp. But there was no noteworthy increase in the fullness of the hair at solutions of 2% and 10% concentrations of peptide copper complex.

Dermal papilla cells (DPCs) are specialized fibroblasts residing at the base of the hair follicle which are important in the morphogenesis and growth of hair follicles. It has been demonstrated that the copper peptide complex L-alanyl-L-histidyl-L-lysine-Cu (AHK-Cu) stimulated the elongation of human hair follicles ex vivo and the proliferation of DPCs in vitro (Pyo H K, Yoo H G, Won C H, Lee S H, Kang Y J, Eun H C et al. The effect of tripeptide-copper complex on human hair growth in vitro. Archives of pharmacal research 2007; Vol.:9). It was concluded that the AHK-Cu promotes the growth of human hair follicles, and this stimulatory effect may occur due to stimulation of the proliferation and the preclusion of the apoptosis of DPCs.

In the fuzzy rat model of hair growth, treatment with a peptide copper complex resulted in an increase in the percent of hair follicles in the anagen or growth phase (Trachy R E, Packard S, Uno H, Pickart L. The fuzzy rat a model of iatrogenic hair growth and the effect of PC-1031. Journal of Investigative Dermatology 39[2], 569a. 1991; and Trachy R E, Uno H, Packard S, Patt L M. Quantitative assessment of peptide-copper complex-induced hair follicle stimulation using the fuzzy rat. In: Maibach H I, editor. Dermatologic research techniques. Boca Raton: CRC Press; 1996. p. 227-39).

In addition, the peptide copper complex caused an increase in hair follicle size, both in terms of the percent of telogen and anagen follicles of longer length and follicle cross-sectional area. These results were consistent with an increase in follicle size due to the prolongation of the anagen phase. The complex caused nearly a 100% increase in the thickness of the hair follicles. Two concentrations of peptide copper complex were tested: one at 2% and the other at 10%.

Eyelashes have a protective function against airborne particles and a cosmetic function to enhance the appearance of the eyes. The loss of fullness or length of the eyelashes can be cosmetically unacceptable to some people while long or full lashes are considered attractive.

There are a number of methods and compositions which have been described for the enhanced growth of hair and in particular eyelashes. For example,

(i) U.S. Pat. No. 5,614,200 discloses a mascara composition having improved application characteristics which would contribute to lash-thickening properties.

(ii) U.S. Patent Application Publication No. 20080275118 discloses compositions and processes for stimulating the growth of hair, including lashes, comprising the application of compositions comprising a hair growth stimulating and/or hair loss prevention agent, and a hair and/or skin lightening and/or neutralization agent. Primarily, the hair growth agents consist of prostaglandins, prostaglandin analogs, 15-PDGH inhibitors, and combinations. This included prostaglandin A2, prostaglandin F2, prostaglandin E1, prostaglandin E2, Arbaprostil, Carboprost, Enprostil, Bimatoprost, Bemeprost, Latanaoprost, Limaprost, Minoxidil, Misoprostol, Ornoprostil, Prostacyclin, Prostaglandin E1, Prostaglandin E2, Prostaglandin F2.alpha., Rioprostil, Rosaprostol, Sulprostone, Travaprost, Trimoprostil, and Viprostol.

(iii) U.S. Patent Application Publication No. 20080269332 discloses effective enhancement of eyelash length and density by topical applications of synthetic prostaglandin agonists, such as 7-[3,5-dihydroxy-2-(3-hydroxy-4-phenoxy-but-1-enyl)-cyclopentyl]-hept-5-enoic acid ethylamide.

(iv) U.S. Patent Application Publication No. 20080241078 discloses a method for treating hair loss including lashes using prostaglandin F analogs.

(v) U.S. Patent Application Publication No. 20080206320 discloses a cosmetic composition containing at least one 15-hydroxy-prostaglandin dehydrogenase inhibitor and cosmetically acceptable excipients for promoting hair growth, including eyelashes.

(vi) U.S. Patent Application Publication No. 20080103184 discloses a method for treating hair loss that uses compositions containing prostaglandin F analogs.

(vii) U.S. Pat. No. 7,351,404 discloses methods and compositions for stimulating the growth of hair including eyelashes which include a cyclopentane heptanoic acid, 2-cycloalkyl or arylalkyl. Bimatoprost ((Z)-7-[(1R,2R,3R,5S)-3,5-Dihydroxy-2-[1E,3S)-3-hydroxy-5-phenyl-1-pentenyl]cyclopentyl]-5-N-ethylheptenamide) is preferred for this treatment.

(viii) U.S. Patent Application Publication No. 20080025940 disclosed 3-Sulfanylpropanamide compounds that are useful in stimulating the growth and density of keratin fibrils or hair.

The above-cited references are incorporated herein by reference in their entireties.

Thus, while there are a number of treatments for eyelashes and eyebrows to enhance the length, thickness, and general cosmetic acceptability currently available, they all are accompanied by various side effects, costs, and/or ineffectiveness. Accordingly, there remains a need in the art for more effective and otherwise improved methods for treatments for eyelashes and eyebrows in need thereof, for example, topically applying compositions, having a desired degree of effectiveness. The present invention fulfills this need and provides further related advantages.

### BRIEF SUMMARY OF THE INVENTION

In brief, the present invention is directed to treating eyelashes and eyebrows by topically applying a composition comprising at least one peptide copper complex to skin at the base of the eyelashes and eyebrows. It has been surprisingly found that such compositions, when topically applied, can increase the length of the treated eyelashes and eyebrows and substantially increase the fullness of the hair, resulting in an enhanced cosmetic appeal. The fullness is increased to a degree more than would be expected from the expected increase in follicle size, and certainly more than would be expected from a solution with a peptide copper complex concentration of 0.2%.

In one representative embodiment, the present invention is directed to a method for treating eyelashes and eyebrows by topically applying to an area of skin at the base of the eyelashes and eyebrows an effective amount of a composition comprising at least one peptide copper complex. Topical application of an effective amount of such a composition substantially increased both the thickness and length of the treated eyelashes and eyebrows resulting in an enhanced cosmetic appeal.

The present invention, in additional related embodiments, is directed to methods for increasing both the thickness and length of the treated eyelashes and eyebrows resulting in an enhanced cosmetic appeal where the topically applied composition used therefor further comprises, in one of the embodiments, an inert and physiologically-acceptable carrier or diluent in addition to the at least one peptide copper complex; and further comprises, in another of the embodiments, a skin conditioning agent, a skin protectant, an emollient, a humectant, or a mixture thereof in addition to the at least one peptide copper complex.

In additional embodiments, disclosed methods of the present invention utilize a composition comprising at least one peptide copper complex, where the composition, in one of the embodiments, further comprises an emulsifying agent, a surfactant, a thickening agent, an excipient, or a mixture thereof; and where the composition, in another of the embodiments, is in the form of a liquid, cream, gel, fluid cream, lotion, emulsion or microemulsion.

These and other aspects of this invention will be evident upon reference to the following detailed description of the invention.

### BRIEF DESCRIPTION OF THE DRAWINGS

FIG. 1 shows the mean percent change in lash length due to application of a peptide copper complex as set forth in Example 2.

FIG. 2 shows the mean difference in lash fullness due to application of a peptide copper complex as set forth in Example 2.

FIG. 3 shows the percentage of subjects showing an increase in lash fullness due to application of a peptide copper complex as set forth in Example 2.

### DETAILED DESCRIPTION OF THE INVENTION

As noted above, in one embodiment, the present invention is directed to treating eyelashes and eyebrows by topically applying a composition comprising at least one peptide copper complex to the skin at the base of the eyelashes and eyebrows. It has been surprisingly found that such compositions, when topically applied, can increase both the fullness and length of the treated eyelashes and eyebrows resulting in an enhanced cosmetic appeal. The increase in fullness is believed to be the result of increased thickness of the hair shafts and in the number of new shafts that may have been spurred from the telogen phase to the anagen phase. Of note is the fact that such results were obtained with a solution having a markedly lesser concentration of the peptide copper complex, i.e. 0.2%.

In more specific embodiments of the disclosed method of the present invention, the composition used therefor comprises at least one peptide copper complex that is L-alanyl-L-histidyl-L-lysine:copper(II) (“AHK-Cu”), L-valyl-L-histidyl-L-lysine:copper(II) (“VHK-Cu”), or glycyl-L-histidyl-L-lysine:copper(II) (“GHK-Cu”).

As used herein, the expression “peptide copper complex” generally refers to a coordination compound comprising a peptide molecule and a copper(II) ion non-covalently complexed therewith. As is well understood in the art, copper (II) designates a copper ion having a valence of 2 (i.e., Cu⁺²). The peptide molecule serves as the complexing agent by donating electrons to the copper ion to yield the non-covalent complex. The peptide molecule is a chain of two or more amino acid units or amino acid derivative units covalently bonded together via amide linkages (for example, —CONH—), the formation of such linkages being accompanied by the elimination of water.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units may be provided by amino acids other than alpha-amino acids. For example, the amino acids may be beta- or gamma-amino acids, such as those shown below.

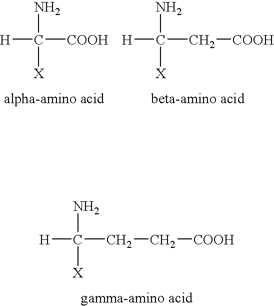

where X is the amino acid side-chain moiety bonded, along with the amino group and hydrogen, to an alpha-, beta-, or gamma-carbon atom.

As another example, the amino acids include, but are not limited to, naturally occurring alpha-amino acids. Naturally occurring amino acids are those from which the amino acid units of naturally occurring proteins are derived. Some of these amino acids, along with their respective amino acid side-chain moieties, are shown below in Table 1. The naturally occurring amino acids shown are all in the L configuration, referring to the optical orientation of the alpha carbon or other carbon atom bearing the amino acid side-chain. A peptide molecule of the present invention may also comprise amino acids that are in the D optical configuration, or a mixture of D and L amino acids.

TABLE 1
Naturally Occurring Amino Acid Side-Chain Moieties

| Amino Acid Side-Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 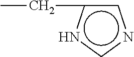 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 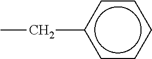 | Phenylalanine |
| 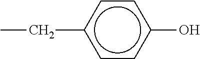 | Tyrosine |
| 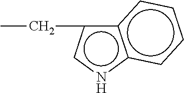 | Tryptophan |
| —CH₂SH | Cysteine |
| —CH₂CH₂SCH₃ | Methionine |
| —CH₂OH | Serine |
| —CH(OH)CH₃ | Threonine |
| 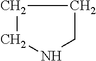 | Proline |

Other naturally occurring amino acids include hydroxyproline and gamma-carboxyglutamate.

Representative amino acid derivatives include those set forth in Table 2 below.

TABLE 2
Amino Acid Derivatives

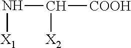

Where X₂ = H or the following moieties:
—(CH₂)ₙCH₃ where n = 1-20
—(CH₂)ₙCH(CH₃)(CH₂)ₘCH₃ where n, m = 0-20 (when n = 0, m ≠ 0 or 1
and when n = 1, m ≠ 0)
—(CH₂)ₙNH₂ where n = 1-20 (n ≠ 4)
—(CH₂)ₙCONH₂ where n = 3-20
—(CH₂)ₙCOOH where n = 3-20 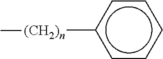
where n = 2-20 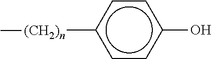
where n = 2-20 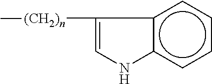
where n = 2-20
—(CH₂)ₙSH where n = 2-20
—(CH₂)ₙS(CH₂)ₘCH₃ where n, m = 1-20 (when n = 2, m ≠ 0)
—(CH₂)ₙCH₂OH where n = 1-20
—(CH₂)ₙCH(CH₃)OH where n = 1-20
And where X₁ = H or the following moieties:
—(CH₂)ₙCH₃ where n = 0-20
—(CH₂)ₙCH(CH₃)(CH₂)ₘCH₃ where n, m = 0-20

Histidine derivatives include compounds having the structure:

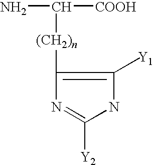

wherein n=1-20, and Y₁ and Y₂ are independently selected from alkyl moieties containing from 1-12 carbon atoms or an aryl moiety containing from 6-12 carbon atoms. In certain embodiments, n is 1, Y₂ is methyl, and Y₁ is H (i.e., 3-methyl histidyl) or Y₂ is H and Y₁ is methyl (i.e., 5-methyl histidine).

Similarly, arginine derivatives include compounds having the structure:

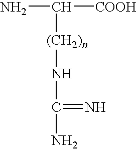

where n=1-20 (excluding n=3).

As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like, while saturated branched alkyls include isopropyl, sec-butyl, isobutyl, tert-butyl, isopentyl, and the like. Representative saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl,” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂-phenyl, —(CH₂)₂-phenyl, —(CH₂)₃-phenyl, —CH(phenyl)₂, and the like).

A peptide copper complex of the present invention may have the formula [R₁-R₂-R₃]:copper(II) where R₃ is at least one amino acid or amino acid derivative, as defined above, bonded to R₂ by a peptide bond (i.e., —C(═O)NH—). Where R₃ is a single amino acid or amino acid derivative, then the peptide of the peptide copper complex is generally classified as a tripeptide. As another example of a peptide copper complex of the present invention having the formula [R₁-R₂-R₃]:copper(II), R₃ is a chemical moiety bonded to the R₂ moiety by an amide bond. The expression “chemical moiety,” as used herein and with reference to R₃, includes any chemical moiety having an amino group capable of forming an amide bond with the carboxyl terminus of R₂ (i.e., the carboxyl terminus of histidine, arginine, or derivatives thereof).

As a more particular example, where R₃ is a chemical moiety bonded to the R₂ moiety by an amide bond, R₃ is —NH₂, an alkylamino moiety having from 1-20 carbon atoms, or an arylamino moiety having from 6-20 carbon atoms. As used herein, an “alkylamino moiety” encompasses alkyl moieties containing an amino moiety, wherein the alkyl moiety is as defined above, and includes, but is not limited to, octyl amine and propyl amine. Similarly, an “arylamino moiety” encompasses aryl moieties containing an amino moiety, wherein the aryl moiety is as defined above, and includes, but is not limited to, benzylamine and benzyl-(CH₂)₁₋₁₄-amine. Further examples of suitable chemical moieties having amino groups capable of forming an amide linkage with the carboxyl terminus of R₂ include polyamines such as spermine and sperimidine.

It should be understood that R₃ may include more than one chemical moiety. For example, additional amino acids or amino acid derivatives may be bonded to the above-described peptide copper complexes comprising tripeptides to yield peptide copper complexes comprising peptides having four or more amino acids and/or amino acid derivatives. For purposes of illustration, Table 3, shown below, presents various representative examples of peptide copper complexes used for or comprised in embodiments of the present invention.

TABLE 3
Representative Peptide-Copper Complexes
Examples of [R₁—R₂]:copper(II)

| | |
|---|---|
| glycyl-histidine:copper | alanyl-histidine:copper |
| glycyl-(3-methyl)histidine:copper | alanyl-(3-methyl)histidine:copper |
| glycyl-(5-methyl)histidine:copper | alanyl-(5-methyl)histidine:copper |
| glycyl-arginine:copper | alanyl-arginine:copper |
| (N-methyl)glycine-histidine:copper | (N-methyl)glycine-arginine:copper |

Examples of [R₁—R₂—R₃]:copper(II)
Where R₃ is Chemical Moiety Linked by Amide Bond

| | |
|---|---|
| glycyl-histidyl-NH₂:copper | glycyl-arginyl-NH₂:copper |
| glycyl-(3-methyl)histidyl-NH₂:copper | alanyl-(3-methyl)histidyl-NH₂:copper |
| glycyl-arginyl-NH₂:copper | alanyl-arginyl-NH₂:copper |
| (N-methyl)glycine-histidyl-NH₂:copper | (N-methyl)glycine-arginyl-NH₂:copper |
| glycyl-histidyl-NHoctyl:copper | glycyl-arginyl-NHoctyl:copper |

Examples of [R₁—R₂—R₃]:copper(II)
where R₃ is Amino Acid or Amino Acid Derivative Linked by Peptide Bond

| | |
|---|---|
| glycyl-histidyl-lysine:copper | glycyl-arginyl-lysine:copper |
| glycyl-(3-methyl)histidyl-lysine:copper | glycyl-(5-methyl)histidyl-lysine:copper |
| alanyl-histidyl-lysine:copper | alanyl-arginyl-lysine:copper |
| alanyl-(3-methyl)histidyl-lysine:copper | alanyl-(5-methyl)histidyl-lysine:copper |
| glycyl-histidyl-phenylalanine:copper | glycyl-arginyl-phenylalanine:copper |
| glycyl-(3-methyl)histidyl- | glycyl-(5-methyl)histidyl- |
| phenylalanine:copper | phenylalanine:copper |
| alanyl-histidyl-phenylalanine:copper | alanyl-arginyl-phenylalanine:copper |
| alanyl-(3-methyl)histidyl- | alanyl-(5-methyl)histidyl- |
| phenylalanine:copper | phenylalanine:copper |
| glycyl-histidyl-lysyl-phenylalanyl- | glycyl-arginyl-lysyl-phenylalanyl- |
| phenylalanyl:copper | phenylalanyl:copper |
| glycyl-(3-methyl)histidyl-lysyl- | glycyl-(5-methyl)histidyl-lysyl- |
| phenylalanyl-phenylalanyl:copper | phenylalanyl-phenylalanyl:copper |
| (N-methyl)glycyl-histidyl-lysine:copper | (N-methyl)glycyl-arginyl-lysine:copper |
| valyl-histidyl-lysine:copper | glycyl-histidyl-lysyl-prolyl-phenylalanyl- |
| prolyl-histidyl-lysine:copper | proline:copper |
| glycyl-D-histidyl-L-lysine:copper | Leucyl-histidyl-lysine:copper |
| seryl-histidyl-lysine:copper | |

Further examples of peptide copper complexes encompassed in embodiments of the present invention are disclosed in U.S. Pat. Nos. 4,665,054; 4,760,051; 4,767,753; 4,810,693; 4,877,770; 5,023,237; 5,059,588; 5,118,665; 5,120,831; 5,164,367; 5,177,061; 5,214,032; 5,538,945; 5,550,183; and 6,017,888, all of which are incorporated herein by reference in their entirety.

Examples of the peptide copper complex derivatives, encompassed in embodiments of the present invention, include, but are not limited to, those disclosed and described in the above-cited U.S. Patents that are directed to peptide copper complexes, as well as those disclosed and described in the published PCT application having the international publication number WO 94/03482, incorporated herein by reference in its entirety.

The synthesis of the above-disclosed peptide copper complexes is described in detail in the above-referenced patents. For example, the peptides of the peptide copper complexes disclosed herein may be synthesized by either solution or solid phase techniques known to one skilled in the art of peptide synthesis. The general procedure involves the stepwise addition of protected amino acids to build up the desired peptide sequence. The resulting peptide may then be complexed to copper (at the desired molar ratio of peptide to copper) by dissolving the peptide in water, followed by the addition of copper chloride or other suitable copper salt and adjusting the pH to greater than 4.0.

Aqueous solutions of peptide copper complexes are prepared by methods that are well known to one skilled in the art. For example, an amount of dried peptide copper complex, suitable for a desired concentration, is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH.

In yet another embodiment of the method of the present invention, the peptide portion of the at least one peptide copper complex used therefor may also be of natural origin. In this embodiment, the peptide is formed by the hydrolysis of naturally occurring proteins, polypeptides, or larger peptides of either plant, microbial, or animal origin. Hydrolysis may be by enzymatic treatment or by acid or base hydrolysis. The copper complex of this type of peptide copper complex is formed by addition of a suitable copper salt to the aqueous solution of the peptide. Alternatively, the peptide copper complex may be formed during the manufacturing of a formulation by separate additions of the peptide and copper salt in a suitable solvent.

In more particular embodiments of the disclosed methods of the present invention, the composition used therefor comprises at least one peptide copper complex where the concentration of the latter, by weight of the composition, ranges from about 0.01% to about 5%; from about 0.025% to about 1%; or from about 0.05% to about 0.5%. In further, more particular embodiments, the molar ratio of peptide to copper in the peptide copper complex ranges from about 1:1 to about 3:1 in one such embodiment, and from about 1:1 to about 2:1 in another such embodiment.

In view of the previously noted beneficial health and cosmetic applications of compositions comprising peptide copper complexes, the compositions used for the methods of the present invention, in certain embodiments, are formulated for use as pharmaceutical or cosmetic products. Accordingly, the method of the present invention, in another embodiment, uses a composition as disclosed above, but further comprising an inert and physiologically-acceptable carrier or diluent.

As noted for the above-disclosed specific embodiment of the method of the present invention, the composition used therefor may comprise a skin conditioning agent. Such an agent typically comprises a substance that adheres to the skin to reduce flaking, restore suppleness, and generally improve the healthy appearance of skin. Representative examples of a skin conditioning agent that may be used include: acetyl cysteine, N-acetyl dihydrosphingosine, acrylates/behenyl acrylate/dimethicone acrylate copolymer, algae extract, allantoin and deriviatives, aloe barbadensis extracts, ceramides, chamomilla recutita (matricaria) flower extract, cycloethoxymethicone, dimethicone copolyols, folic acid, gelatin, beta-glucan, hydrogenated proteins, hydrolyzed proteins, jojoba oil, keratin, keratin amino acids, and kinetin.

Other non-limiting examples of skin conditioning agents that may be included in the compositions used for the present invention are: lecithin, linoleic acid, linolenic acid, malt extract, maltodextrin, oat amino acids, oryzanol, palmitoyl hydrolyzed proteins, phytosterols, riboflavin, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. A skin conditioning agent, other than those listed above, may also be used, as is readily appreciated by those skilled in the art.

A skin protectant, for purposes of the present invention, refers to a compound that protects injured or exposed skin or mucous membrane surfaces from harmful or irritating external compounds. Representative examples thereof include: algae extract, allantoin, aluminum hydroxide, aluminum sulfate, betaine, camellia sinensis leaf extract, cerebrosides, dimethicone, glucuronolactone, glycerin, kaolin, lanolin, malt extract, mineral oil, petrolatum, potassium gluconate, and talc. One skilled in the art will readily appreciate that skin protectants other than those listed above may also be combined with a disclosed composition of the present invention or preparation provided thereby.

An emollient, as the term is used herein, is a cosmetic ingredient that can help skin at the base of the eyelashes and eyebrows maintain a soft, smooth, and pliable appearance. Emollients are able to provide these benefits, largely owing to their ability to remain on the skin surface, or in the stratum corneum, to act as a lubricant and reduce flaking. Some examples of an emollient, suitable for use in the above-disclosed, specific embodiment of this invention, are: acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, C₁₈-C₃₆ acid glycol ester, C₁₂-C₁₃ alkyl lactate, caprylyl glycol, cetyl esters, cetyl laurate, coconut oil polyethylene glycol-10 esters, hexyl isostearate, hydrogenated palm glycerides, hydrogenated soy glycerides, hydrogenated tallow glycerides, isostearyl neopentanoate, isostearyl palmitate, isotridecyl isononanoate, mineral oil, palm glycerides, polyethylene glycol avocado glycerides, polyethylene glycol castor oil, polyethylene glycol shorea butter glycerides, phytol, raffinose, stearyl citrate, sunflower seed oil glycerides, and tocopheryl glucoside. Those skilled in the art will readily appreciate that emollients, other than those listed above, may also be used.

Humectants are cosmetic ingredients that help maintain moisture levels in skin at the base of the eyelashes and eyebrows. Some examples of suitable humectants are: acetyl arginine, algae extract, aloe barbadensis leaf extract, diglycerin, fructose, glucose, glycerin, honey, hydrolyzed wheat protein/polyethylene glycol-20 acetate copolymer, inositol, lactitol, maltitol, maltose, mannitol, mannose, propylene glycol, sodium PCA, sorbitol, sucrose, and urea. Other humectants may be used for yet additional embodiments of this invention, as will be appreciated by those skilled in the art.

In addition to the active ingredients described above, the disclosed compositions and preparations provided thereby may also contain inert, physiologically acceptable carriers or diluents. Suitable carriers or diluents include, but are not limited to: water, physiological saline, bacteriostatic saline (e.g., saline containing 0.9 mg/ml benzyl alcohol), petrolatum based creams (e.g., USP hydrophilic ointments and similar creams), various types of pharmaceutically acceptable gels, and short chain alcohols and glycols (e.g., ethyl alcohol and propylene glycol).

In a further specific embodiment of the method of the present invention, the composition used therefor comprises a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof. Non-limiting examples of a suitable phospholipid include lecithin and cephalin. Suitable hydrocarbon oils include, but are not limited to, palm oil, coconut oil, and mineral oil.

Emulsifiers and surfactants are used in preparing embodiments of the present invention directed to compositions and preparations formulated as emulsions. Either water in oil or oil in water emulsions may be formulated. Examples of suitable surfactants and emulsifying agents include: nonionic ethoxylated and nonethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, ceteareth-7, cetyl alcohol, cetyl phosphate, corn oil PEG esters, DEA-cetyl phosphate, dextrin laurate, dilaureth-7 citrate, dimyristyl phosphate, glycereth-17 cocoate, glyceryl erucate, glyceryl laurate, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPG's, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths. Other surfactants and emulsifiers may be used, as will be appreciated by one skilled in the art.

Examples of a thickening (i.e., viscosity increasing) agent that is suitable for inclusion in the composition used for the above-disclosed embodiment, include, but are not limited to, those agents commonly used in skin care or lash care preparations. More specifically, such examples include acrylamides copolymer, agarose, amylopectin, carbomer, cellulose gum, dextrin, gelatin, hydroxyethylcellulose, hydroxypropylcellulose, hydroxypropyl starch, methylcellulose, microcrystalline cellulose, pectin, various polyethylene glycol's, polyacrylic acid, polymethacrylic acid, polyvinyl alcohol, various PPG's, sodium acrylates copolymer, sodium carrageenan, xanthan gum, and yeast beta-glucan. Thickening agents other than those listed above may also be used in related embodiments of the present invention.

Excipients may also be combined with disclosed compositions and preparations. A suitable excipient is adapted for application to the face and neck. More specifically, a suitable excipient should have a high affinity for the skin, be well tolerated, stable, and yield a consistency that allows for easy and pleasant utilization.

The compositions of the present invention, as well as the pharmaceutical and cosmetic preparations for skin provided thereby, are intended primarily as products for topical application to human skin. Accordingly, in one embodiment the disclosed composition is in the form of a liquid, cream, gel, fluid cream, lotion, emulsion or microemulsion.

Typically, for a method of the present invention, a small amount of the composition used therefor is applied to exposed areas of skin at the base of the eyelashes and eyebrows from a suitable container or applicator. Each composition disclosed herein is typically packaged in a container that is appropriate in view of its viscosity and intended use by the consumer.

The following examples are provided for the purpose of illustration, not limitation.

### EXAMPLES

The examples which follow illustrate the preparation, characterization and utility of certain compositions used for exemplary embodiments of the methods of the present invention, and illustrate the effectiveness of such methods in increasing both the thickness and length of the treated eyelashes resulting in an enhanced cosmetic appeal.

### Example 1

A typical formulation containing the peptide copper complex L-Alanyl-L-Histidyl-L-Lysine Copper for treating eyelashes was prepared as follows below.

| Ingredient | % |
|---|---|
| Water | Greater than 50% |
| TEA-Carbomer | Less than 2% |
| Panthenol | Less than 2% |
| Sodium Hyaluronate | Less than 1% |
| Phenoxyethanol | 0.90% |
| Hydrolyzed Glycosaminoglycans | Less than 1% |
| Sodium Benzoate | 0.25% |
| Dimethicone Copolyol | Less than 1% |
| Guar Hydroxypropyltrimonium | Less than 1% |
| Chloride | |
| Hydrolyzed Keratin | Less than 1% |
| Ethylhexylglycerin | Less than 1% |
| L-Alanyl-L-Histidyl-L-Lysine Copper | 0.20% |

### Example 2

The utility of a composition used for a method of the present invention was demonstrated in an 8 week study involving 26 subjects. The composition of Example 1 was applied as a thin line with a brush applicator to the upper eyelid on one eye nightly for up to 8 weeks. At the study initiation, week 3, 6, and 8, lash length and fullness was evaluated from high resolution photographs. The other eyelid was left untreated. At the completion of the study, the lash length and fullness was determined and evaluated from the high resolution photographs.

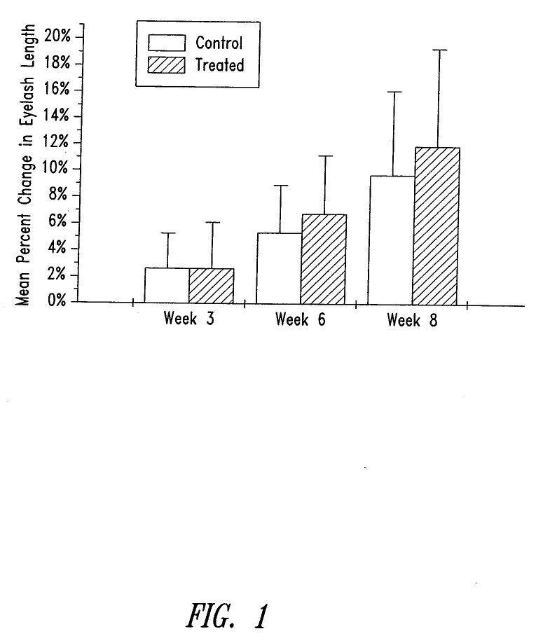

FIG. 1 shows the mean percent change in lash length in both groups throughout the study duration. The error bars are the standard deviation of the mean.

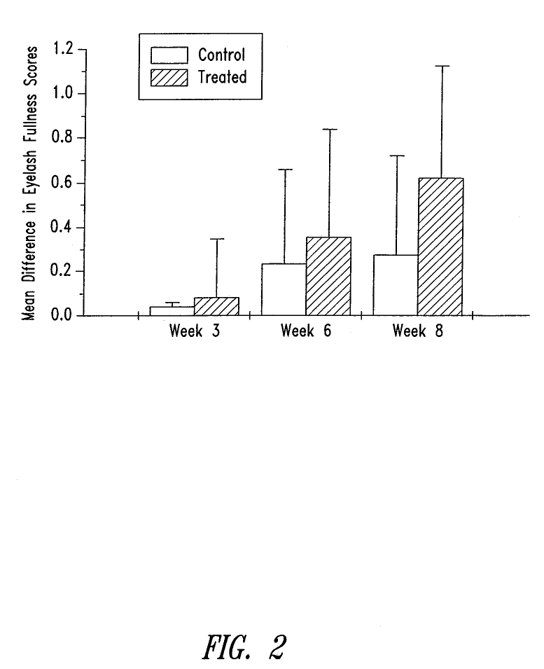

Lash fullness was determined on a 9 point scale at each interval by an evaluator blinded as to the treatment. FIG. 2 shows the mean difference in lash fullness. At week 8, the difference is significant at p=0.0012. The error bars are the standard deviation of the mean. By week 8 the fullness of the lash was more than 100% of the fullness of the control group.

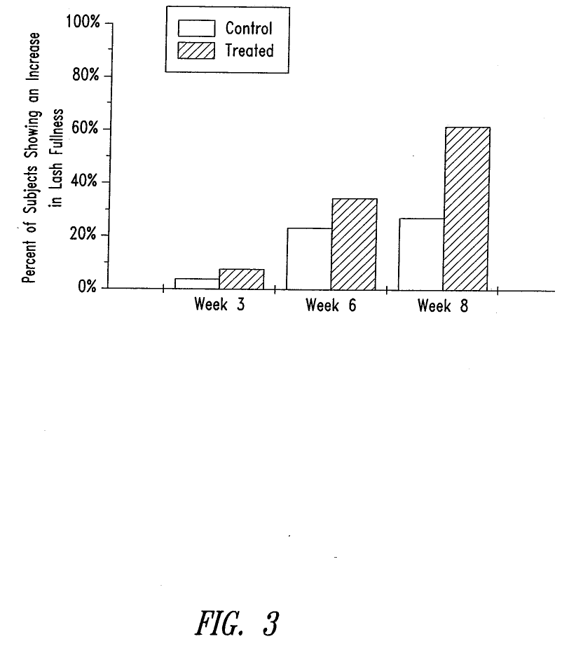

FIG. 3 shows the percentage of each treatment group which had an increase in the lash fullness score by week 8.62% of the subjects treated with the formulation of Example 1 showed an increase in lash fullness. By week 8, the fullness was more than 100% of that of the control group.

Thus, the above examples demonstrate the effectiveness of the composition and method of the present invention in increasing both the fullness and length of the treated eyelashes resulting in an enhanced cosmetic appeal.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the present invention have been described herein for purposes of illustration, various modifications may be made without deviating from the spirit and scope of the invention. Accordingly, the present invention is not limited except as by the appended claims.

What is claimed is:

1. A method for enhancing the fullness and length of eyelashes and eyebrows comprising topically applying to an area of skin at the base of the eyelashes and eyebrows an effective amount of a composition, wherein the composition comprises at least one peptide copper complex.

2. The method of claim 1 wherein the at least one peptide copper complex is L-alanyl-L-histidyl-L-lysine:copper(II).

3. The method of claim 1 wherein the at least one peptide copper complex is L-valyl-L-histidyl-L-lysine:copper(II).

4. The method of claim 1 wherein the at least one peptide copper complex is glycyl-L-histidyl-L-lysine:copper(II).

5. The method of claim 1 wherein the at least one peptide copper complex is comprised of a peptide formed by the hydrolysis of naturally occurring proteins, polypeptides, and larger peptides of either plant, microbial, or animal origin.

6. The method of claim 1 wherein the molar ratio of peptide to copper in the at least one peptide copper complex ranges from about 1:1 to about 3:1.

7. The method of claim 1 wherein the molar ratio of peptide to copper in the at least one peptide copper complex ranges from about 1:1 to about 2:1.

8. The method of claim 1 wherein the at least one peptide copper complex is present at a concentration ranging from about 0.01% to about 5% by weight of the composition.

9. The method of claim 1 wherein the at least one peptide copper complex is present at a concentration ranging from about 0.025% to about 1% by weight of the composition.

10. The method of claim 1 wherein the at least one peptide copper complex is present at a concentration ranging from about 0.05% to about 0.5% by weight of the composition.

11. The method of claim 1 wherein the composition further comprises a physiologically-acceptable carrier or diluent.

12. The method of claim 11 wherein the inert and physiologically-acceptable carrier or diluent is water, physiological saline, bacteriostatic saline, a petrolatum based cream, a pharmaceutically acceptable gel, a short chain alcohol, or a short chain glycol.

13. The method of claim 1 wherein the composition further comprises a skin conditioning agent, a skin protectant, an emollient, a humectant, or a mixture thereof.

14. The method of claim 1 wherein the composition further comprises an emulsifying agent, a surfactant, a thickening agent, an excipient, or a mixture thereof.

15. The method of claim 1 wherein the composition is in the form of a liquid, cream, gel, fluid cream, lotion, emulsion or microemulsion.

16. A composition for enhancing the fullness and length of eyelashes and eyebrows comprising at least one peptide copper complex.

17. The composition of claim 16 wherein the at least one peptide copper complex is L-alanyl-L-histidyl-L-lysine:copper(II).

18. The composition of claim 16 wherein the at least one peptide copper complex is L-valyl-L-histidyl-L-lysine:copper(II).

19. The composition of claim 16 wherein the at least one peptide copper complex is glycyl-L-histidyl-L-lysine:copper(II).

20. The composition of claim 16 wherein the at least one peptide copper complex is comprised of a peptide formed by the hydrolysis of naturally occurring proteins, polypeptides, and larger peptides of either plant, microbial, or animal origin.

21. The composition of claim 16 wherein the molar ratio of peptide to copper in the at least one peptide copper complex ranges from about 1:1 to about 3:1.

22. The composition of claim 16 wherein the molar ratio of peptide to copper in the at least one peptide copper complex ranges from about 1:1 to about 2:1.

23. The composition of claim 16 wherein the at least one peptide copper complex is present at a concentration ranging from about 0.01% to about 5% by weight of the composition.

24. The composition of claim 16 wherein the at least one peptide copper complex is present at a concentration ranging from about 0.025% to about 1% by weight of the composition.

25. The composition of claim 16 wherein the at least one peptide copper complex is present at a concentration ranging from about 0.05% to about 0.5% by weight of the composition.

26. The composition of claim 16 further comprising a physiologically-acceptable carrier or diluent.

27. The composition of claim 26 wherein the inert and physiologically-acceptable carrier or diluent is water, physiological saline, bacteriostatic saline, a petrolatum based cream, a pharmaceutically acceptable gel, a short chain alcohol, or a short chain glycol.

28. The composition of claim 16 further comprising a skin conditioning agent, a skin protectant, an emollient, a humectant, or a mixture thereof.

29. The composition of claim 16 further comprising an emulsifying agent, a surfactant, a thickening agent, an excipient, or a mixture thereof.

30. The composition of claim 16 wherein the composition is in the form of a liquid, cream, gel, fluid cream, lotion, emulsion or microemulsion.
